SEQUENCE LISTING (8 sequences)

SEQ ID NO: 1 - DNA, 20 bp, H. capsulatum
ccttgtctaccggacctgtt

SEQ ID NO: 2 - DNA, 20 bp, H. capsulatum
ttcttcatcgatgtcggaac

SEQ ID NO: 3 - DNA, 16 bp, H. capsulatum
ttgtctaccggacctg

SEQ ID NO: 4 - DNA, 20 bp, H. capsulatum
ttcttcatcgatgtcggaac

SEQ ID NO: 5 - DNA, 21 bp, H. capsulatum
ggtgtacgattggcctctgag

SEQ ID NO: 6 - DNA, 25 bp, H. capsulatum
tgagagcgataataatccagtcgag

SEQ ID NO: 7 - DNA, 17 bp, H. capsulatum
acgattggcgtctgagc

SEQ ID NO: 8 - DNA, 26 bp, H. capsulatum
gagagcgataataatccagtcaaaac

(19) United States
(12) Patent Application Publication
(10) Pub. No.: US 2005/0065330 A1
(43) Pub. Date: Mar. 24, 2005

(54) Identification of Histoplasma capsulatum using a PCR assay

(75) Inventor: Gary W. Procop, Twinsburg, OH (US)

Correspondence Address: TAROLLI, SUNDHEIM, COVELL & TUMMINO L.L.P., SUITE 1111, 526 SUPERIOR AVENUE, CLEVELAND, OH, 44114-1400, US

(73) Assignee: The Cleveland Clinic Foundation

(21) Appl. No.: 10/421,196

(22) Filed: Apr. 23, 2003

Related U.S. Application Data
(60) Provisional application No. 60/375,601, filed on Apr. 24, 2002.

(51) Int. Cl. C07H 21/02; C07H 21/04

(52) U.S. Cl. 536/23.1

(57) ABSTRACT

A nucleic acid specific for use in identifying Histoplasma capsulatum comprises a sequence selected from the group consisting of SEQ ID NOs: 1-8, a sequence complementary to SEQ ID NOs: 1-8, a sequence substantially similar to SEQ ID NOs: 1-8, a sequence substantially similar to a sequence complementary to SEQ ID NOs: 1-8, and a fragment of SEQ ID NOs: 1-8, a sequence complementary to SEQ ID NOs: 1-8, or a sequence substantially similar to SEQ ID NOs: 1-8, a sequence substantially similar to a sequence complementary to SEQ ID-NO: 1-8 that specifically hybridizes to Histoplasma capsulatum.

### FIELD OF THE INVENTION

The present invention relates to the detection and the identification of the pathogenic fungus, Histoplasma capsulatum. In particular, the present invention relates to nucleic acid sequences for oligonucleotide primers and hybridization probes that may be used in the detection and identification of Histoplasma capsulatum by means of a polymerase chain reaction (PCR) assay.

### BACKGROUND OF THE INVENTION

Histoplasma capsulatum is a slow-growing dimorphic fungal pathogen, which under different environmental conditions, can exist in either a yeast or mold form. The yeast form is unicellular and reproduces by budding on specialized media at 37° C. The mold form produces multi-cellular filamentous colonies that consist of cylindrical structures referred to as hyphae. The mold form may further contain macroconidia and microconidia, which primarily grow under appropriate soil conditions or on specialized fungal media at 25° C.

In humans, H. capsulatum can cause the systemic fungal disease, histoplasmosis. The symptoms vary from those of a mild, self-limited infection to a severe or fatal disease. Symptoms of the severe form of histoplasmosis are fever, anemia, enlarged spleen and liver, leukopenia, pulmonary involvement, adrenal necrosis, and gastrointestinal ulcers. Immuno-compromised patients, particularly the elderly and those who have acquired immune deficiency syndrome (AIDS), are especially susceptible to histoplasmosis.

It is important to properly identify H. capsulatum from other fungal species in order to determine proper treatment of an infection. One conventional method for isolating and identifying H. capsulatum uses a culture identification assay. In a culture identification assay, a clinical specimen of an organism suspected of being H. capsulatum is first grown in culture. After culturing the organism, the DNA or RNA of the cultured organism is detected and identified with an oligonucleotide probe. An example of a culture identification assay for H. capsulatum is ACCUPROBE, which is commercially available from Gen-Probe Inc., of San Diego, Calif.

Another method for detecting and identifying H. capsulatum uses a nucleic acid amplification assay for either direct amplification of the ribosomal DNA (rDNA) genes by polymerase chain reaction or reverse transcription of ribosomal RNA (rRNA) into complementary DNA (cDNA) followed by polymerase chain reaction amplification of the cDNA. The amplification product, i.e., amplicon, is then detected by probes or other mechanical means to definitely identify the DNA.

U.S. Pat. No. 5,352,579 describes nucleic acid hybridization assay probes, which are specific to H. capsulatum and no other fungi and which have the nucleic sequence 5′-CGAAGTCGAGGCTTTCAGCATG-3′, or the nucleotide sequence complementary thereto. A probe having the above nucleic acid sequence hybridizes to the 18S rRNA of H. capsulatum corresponding to bases 172-193 of Sacchromyces cerevisiae. This patent also describes the use of helper probes having the sequence 5′-TATTAGCTCTAGAATTACCAGGGTATCCAAGTAGTAAGG-3′, or the sequence 5′-CCCCGAAGGGCATTGGTTTTTTTATCTAATAAATACACCCC-3′.

U.S. Pat. No. 5,693,501 describes nucleic acid sequences from the internal transcribed spacer region 1 (ITS-1) of Histoplasma capsulatum. The nucleic acid sequences are used to design a PCR method that allows specific detection and identification of H. capsulatum. Preferably, the nucleic acid sequences are used as primers to amplify a section of the ITS-1 region of H. capsulatum. Three sets of primers are disclosed. The nucleic acid sequences for the three sets of primers have the following respective sequences:

| | |
|---|---|
| 1) | 5′-TGCGGAAGGATCATTACCACGC-3′ |
| and | |
| | 5′-ATCGCTCTCATGCTCAGACGCC-3′; |
| 2) | 5′-CGGAAGGATCATTACCACGCCG-3′ |
| and | |
| | 5′-TCGTTCACCGACGGTTCTTACGG-3′; |
| 3) | 5′-AAGCTGGTCAAACTTGGTC-3′ |
| and | |
| | 5′-AGATCCGTTGTTGAAAGTTT-3′. |

WO 99/54508 describes nucleic acid sequences that are useful as primers to initiate the amplification of a segment of Histoplasma capsulatum DNA, which is specific to the 5.8s rRNA gene of H. capsulatum, using PCR. The primers include the sequence 5′-GGAGCCTCTGACCGGGAC-3′ and 5′-GCACGTCCCACCGGTCAG-3′.

### SUMMARY OF THE INVENTION

The present invention relates to a nucleic acid specific for use in identifying Histoplasma capsulatum. The nucleic acid comprises a sequence that is selected from the group consisting of SEQ ID NOs: 1-8, a sequence complementary to SEQ ID NOs: 1-8, a sequence substantially similar to SEQ ID NOs: 1-8, a sequence substantially similar to a sequence complementary to SEQ ID NOs: 1-8, and a fragment of SEQ ID NOs: 1-8, a sequence complementary to SEQ ID NOs: 1-8, or a sequence substantially similar to SEQ ID NOs: 1-8, a sequence substantially similar to a sequence complementary to SEQ ID NO: 1-8 that specifically hybridizes to Histoplasma capsulatum.

Another aspect of the present invention relates to a oligonucleotide primer specific for use in identifying Histoplasma capsulatum. The oligonucleotide primer comprises a nucleic acid sequence selected from the group consisting of SEQ ID NOs: 1-4, a sequence complementary to SEQ ID NOs: 1-4, a sequence substantially similar to SEQ ID NOs: 1-4, a sequence substantially similar to a sequence complementary to SEQ ID NOs: 1-4, and a fragment of SEQ ID NOs: 1-4, a sequence complementary to SEQ ID NOs: 1-4, or a sequence substantially similar to SEQ ID NOs: 1-4, a sequence substantially similar to a sequence complementary to SEQ ID NO: 1-4 that specifically hybridizes to Histoplasma capsulatum.

A further aspect of the present invention relates to an oligonucleotide hybridization probe for identifying Histoplasma capsulatum. The probe comprises a nucleic acid sequence selected from the group consisting of SEQ ID NOs: 5-8, a sequence complementary to SEQ ID NOs: 5-8, a sequence substantially similar to SEQ ID NOs: 5-8, a sequence substantially similar to a sequence complementary to SEQ ID NOs: 5-8, and a fragment of SEQ ID NOs: 5-8, a sequence complementary to SEQ ID NOs: 5-8, or a sequence substantially similar to SEQ ID NOs: 5-8, a sequence substantially similar to a sequence complementary to SEQ ID NOs: 5-8 that specifically hybridizes to Histoplasma capsulatum.

The present invention also relates to a method of detecting the presence of Histoplasma capsulatum in a sample. In the method, a sample suspected of including Histoplasma capsulatum is provided. A Histoplasma capsulatum target nucleotide sequence is amplified using an oligonucleotide primer comprising a nucleic acid sequence selected from the group consisting of SEQ ID NOs: 1-4, a sequence complementary to SEQ ID NOs: 1-4, a sequence substantially similar to SEQ ID NOs: 1-4, a sequence substantially similar to a sequence complementary to SEQ ID NOs: 1-4, and a fragment of SEQ ID NOs: 1-4, a sequence complementary to SEQ ID NOs: 1-4, on a sequence substantially similar to SEQ ID NOs: 1-4, a sequence substantially similar to a sequence complementary to SEQ ID NO: 1-4 that specifically hybridizes to Histoplasma capsulatum. The amplified target nucleic acid is detected with an oligonucleotide hybridization probe, which is capable of hybridizing to the amplified target nucleotide sequence.

In another method of detecting the presence of Histoplasma capsulatum in a sample, a sample suspected of including Histoplasma capsulatum is provided. A target nucleotide sequence that comprises contiguous nucleotides from the ITS-1 and 5.8s gene region of Histoplasma capsulatum is amplified. The amplified target nucleotide sequence is contacted with an oligonucleotide hybridization probe comprising a nucleic acid sequence selected from the group consisting of SEQ ID NOs: 5-8, a sequence complementary to SEQ ID NOs: 5-8, a sequence substantially similar to SEQ ID NOs: 5-8, a sequence substantially similar to a sequence complementary to SEQ ID NOs: 5-8, and a fragment of SEQ ID NOs: 5-8, a sequence complementary to SEQ ID NOs: 5-8, or a sequence substantially similar to SEQ ID NOs: 5-8, a sequence substantially similar to a sequence complementary to SEQ ID NOs: 5-8 that specifically hybridizes to Histoplasma capsulatum.

A further aspect of the present invention relates to a kit for use in detecting Histoplasma capsulatum. The kit comprises a primer that includes a nucleic acid sequence selected from the group consisting of SEQ ID NOs: 1-4, a sequence complementary to SEQ ID NOs: 1-4, a sequence substantially similar to SEQ ID NOs: 1-4, a sequence substantially similar to a sequence complementary to SEQ ID NOs: 1-4, and a fragment of SEQ ID NOs: 1-4, a sequence complementary to SEQ ID NOs: 1-4, or a sequence substantially similar to SEQ ID NOs: 1-4, a sequence substantially similar to a sequence complementary to SEQ ID NO: 1-4 that specifically hybridizes to Histoplasma capsulatum. The kit further includes a nucleic acid hybridization probe that includes a nucleic acid sequence selected from the group consisting of SEQ ID NOs: 5-8, a sequence complementary to SEQ ID NOs: 5-8, a sequence substantially similar to SEQ ID NOs: 5-8, a sequence substantially similar to a sequence complementary to SEQ ID NOs: 5-8, and a fragment of SEQ ID NOs: 5-8, a sequence complementary to SEQ ID NOs: 5-8, or a sequence substantially similar to SEQ ID NOs: 5-8, a sequence substantially similar to a sequence complementary to SEQ ID NOs: 5-8 that specifically hybridizes to Histoplasma capsulatum.

### DESCRIPTION OF EMBODIMENTS

The present invention may be understood more readily by reference to the following detailed description of the embodiments of the invention, and to the Examples and Sequence listings included therein.

As used herein in the specification and the claims, the following terms have the given meaning unless expressly stated to the contrary.

A “nucleotide” is a subunit of a nucleic acid consisting of a phosphate group, a 5-carbon sugar and a nitrogenous base. In DNA, the 5-carbon sugar is deoxyribose. For a 5′-oligonucleotide, the sugar contains a hydroxyl group (—OH) at the 5′ carbon.

The phrase “specific to” and “unique to” the fungus H. capsulatum as used herein in relation to a nucleic acid or nucleic acid fragment means a nucleic acid or nucleic acid fragment that is not common to other related fungi or other microorganisms (i.e., it is only present in the fungus H. capsulatum).

The phrase “sample” as used herein means any sample of fluid, or of solubized or nonsolubized tissue obtained from a subject, or solubized or nonsolubized cultured cells, which contains components, such as nucleic acids or fragments thereof, that may be employed in one of the tests described herein to detect a previous or current infection by, or exposure to, the fungus H. capsulatum, or to make a positive diagnosis of histoplasmosis. Such samples include blood, serum, plasma, sputum, urine, mucus, saliva, gastric juice, lymph, feces, or other bodily fluids, and tissues from the lungs, spleen, liver, skin, or other organs. The samples can also be supernatant from incubated tissue sample or cultured cells.

The term “fragment” as used herein in relation to a nucleic acid means a sub-sequence of a nucleic acid that is of a sufficient size and confirmation to properly function as a hybridization probe or as a primer in a polymerase chain reaction or in another manner characteristic of nucleic acids.

The term “hybridization” as used herein refers to the formation of a duplex structure by two single-stranded nucleic acids due to fully (100%) or less than fully (less than 100%) complementary base pairing. Hybridization can occur between fully and complementary nucleic acid strands, or between less than fully complementary nucleic acid strands which contain regions of mismatch due to one or more nucleotide substitutions, deletions, or additions.

The term “isolated” means that the nucleic acids or nucleic acid fragments are of sufficient purity so that they may be employed, and will function properly, in a clinical diagnostic, experimental or other procedure, such as a hybridization assay or an amplification reaction for histoplasmosis. Many procedures are known by those of ordinary skill in the art for purifying nucleic acids, nucleic acid fragments, and materials with which they may normally be associated prior to their use in various procedures.

The term “substantially similar” in relation to the nucleic acid sequences of SEQ ID NOs: 1-8, or to the nucleotide sequences complementary to SEQ ID NOs: 1-8, refers to a nucleic acid which is similar to the to the nucleic acid sequence set forth in SEQ ID NOs: 1-8, or to nucleic acid sequences complementary to SEQ ID NOs: 1-8, and which retains the functions of such nucleic acid, but which differs from such nucleic acid by the substitution, deletion, and/or addition of one or more nucleotides, and/or by the incorporation of some other advantageous feature. Nucleotide sequences of the present invention are substantially similar to a nucleic acid sequence if these percentages are from 100% to 80% or from 0 base mismatches in a 10 nucleotide sequence to 2 bases mismatched in a 10 nucleotide sequence. In some embodiments, the percentage is from 100% to 85%. In other embodiments, this percentage is from 90% to 100%; in still other embodiments, this percentage is from 95% to 100%.

The phrase “target nucleotide sequence” refers to a region of a nucleotide which is to be amplified, detected, or otherwise analyzed. The sequence to which the oligonucleotide probe hybridizes is referred to as a target nucleotide sequence.

The present invention relates to nucleic acid sequences that can be used for the detection and identification of Histoplasma capsulatum. Particularly, the nucleic acid sequences can be used as oligonucleotide primers and hybridization probes for the detection and identification of H. capsulatum.

The oligonucleotide primers of the present invention serve as a priming position or initiation position for the action of primer dependent DNA polymerase activity. The oligonucleotide primers include nucleic acid sequences that are specific to H. capsulatum and that can be used to amplify a target nucleotide sequence. The target nucleotide sequence is defined by contiguous nucleotides from the ITS-1 region and 5.8s gene region of H. capsulatum. The contiguous nucleotides of the ITS-1 and 5.8s region include contiguous nucleotides to which the oligonucleotide hybridization probes of the present invention can hybridize.

The oligonucleotide primers of the present invention can include a nucleic acid sequence selected from the group consisting of SEQ ID NOs: 1-4, a nucleic acid sequence complementary to SEQ ID NOs: 1-4, a nucleic acid sequence substantially similar to SEQ ID NOs: 1-4, a nucleic acid sequence substantially similar to a nucleic acid sequence complementary to SEQ ID NOs: 1-4, a fragment of SEQ ID NOs: 1-4 that specifically hybridize to H. capsulatum, a fragment of a nucleic acid sequence complementary to SEQ ID NOs: 1-4 that specifically hybridize to H. capsulatum, a fragment of a nucleic acid sequence substantially similar to SEQ ID NOs: 1-4 that specifically hybridizes to H. capsulatum, and a fragment of a nucleic acid sequence substantially similar to nucleic acid sequences complementary to SEQ ID NOs: 1-4 that specifically hybridizes to H. capsulatum.

Preferably, the oligonucleotide primers of the present invention comprise a pair of oligonucleotide primers that hybridize to nucleotide sequences, which flank the target nucleotide sequence, so that DNA synthesis by the action of a DNA polymerase, such as Taq polymerase, proceeds through the region between the two primers. This is advantageous because after several rounds of hybridization and replication the amplified target nucleotide sequence produced is a segment having a defined length whose ends are defined by the sites to which the primers hybridize.

A preferred pair of nucleic acid sequences that can be used for the pair of oligonucleotide primers include SEQ ID NOs: 1 and 2. Another preferred pair of nucleic acid sequences that can be used for the pair of oligonucleotide primers include SEQ ID NOs: 3 and 4. Pairs of nucleic acid sequences that are complementary and/or substantially similar to SEQ ID NOs: 1 and 2 and SEQ ID NOs: 3 and 4, are also preferred.

The oligonucleotide hybridization probes in accordance with the present invention are used to detect the target nucleotide sequence amplified by the oligonucleotide primers of the present invention. The oligonucleotide hybridization probes include a nucleic acid sequence that is capable of hybridizing to the amplified target amplified target nucleotide sequence of H. capsulatum. Preferred oligonucleotide probes that can hybridize to the amplified target nucleotide sequence include at least one nucleic acid sequence selected from the group consisting of SEQ ID NOs: 5-8, a nucleic acid sequence complementary to SEQ ID NOs: 5-8, a nucleic acid sequence substantially similar to SEQ ID NOs: 5-8, a nucleic acid sequence substantially similar to nucleic acid sequences complementary to SEQ ID NOs 5-8, a fragment of SEQ ID NOs: 5-8 that specifically hybridize to H. capsulatum, a fragment of a nucleic acid sequence complementary to SEQ ID NOs: 5-8 that specifically hybridize to H. capsulatum, a fragment of a nucleic acid sequence substantially similar to SEQ ID NOs: 5-8 that specifically hybridizes to H. capsulatum, and a fragment of a nucleic acid sequence substantially similar to a nucleic acid sequences complementary to SEQ ID NOs 5-8 that specifically hybridizes to H. capsulatum.

The oligonucleotide hybridization probes of the present invention are preferably labeled with a detectable moiety, which can be used to detect or confirm hybridization of the oligonucleotide hybridization probes to their target sequence. The detectable moiety can be a molecule that is attached to, or synthesized as part of the oligonucleotide hybridization probe. The molecule should be uniquely detectable and allow the oligonucleotide hybridization probes to be detected as a result. Examples of detectable moieties include isotopic labels, radioactive labels, biotin, enzymes, digoxigenin, chemiluminescent labels, and fluorescent labels. The detection method selected will depend upon the hybridization conditions and detectable moiety used for labeling.

In a preferred embodiment of the present invention, fluorescence resonance energy transfer (FRET) is used to detect the oligonucleotide hybridization probes. For this detection format, two oligonucleotide hybridization probes are used that are capable of hybridizing in head-to-tail arrangement to adjacent but non-overlapping regions of the amplified target nucleotide acid sequence of H. capsulatum. The two oligonucleotide hybridization probes are each labeled with a respective member of fluorescent resonance energy transfer pair. One oligonucleotide hybridization probe is labeled at the 3′-end with a donor fluorophore, and the other oligonucleotide hybridization probe is labeled at the 5′-end with an acceptor fluorophore.

Fluorophore pairs that can be used as fluorescence resonance energy transfer pairs are well known to those skilled in the art. A preferred donor fluorophore is fluorescein (5-FITC), which is commercially available from Synthegen, LLC of Houston, Tex. The 3′-end of the one oligonucleotide probe can be labeled with fluorescein (5-FITC) by using a dye-derived, controlled pore glass or by post-labeling the 3′-amino modified oligonucleotide. An example of preferred acceptor fluorophore is LightCycler-Red 640 NHS ester, which is commercially available from Synthegen, LLC. The 5′-end of the other oligonucleotide probe can be labeled with LightCycler-Red 640 NHS ester by reaction of the LightCycler-Red 640 NHS ester with a 5′-amino-modified oligonucleotide in a sodium borate buffered solution. Examples of other commercially available donor/acceptor fluorophore pairs include fluorescein/LightCycler Red 705, fluorescein/Cy7, fluorescein/Cy5, and fluorescein/Cy5.5, all of which are commercially available from Synthegen, LLC of Houston, Tex.

If the amplified target nucleotide sequence is present, the fluorescently labeled oligonucleotide hybridization probes hybridize to the amplified target nucleotide sequence resulting in the donor and the acceptor fluorophores being separated by a distance of about 0-5 nucleotides, or more preferably 0-2 nucleotides.

Fluorescent resonance energy transfer (FRET) occurs between the donor fluorophore and acceptor fluorophore when they are in physical proximity to one another so that the donor fluorophore can transfer resonance energy to the acceptor fluorophore and the acceptor fluorophore can produce a measurable fluorescence emission. As a consequence, the hybridization can be monitored through excitation of the donor fluorophore and subsequent measurement of the fluorescence emission of the acceptor fluorophore.

When both the fluorescently labeled oligonucleotide probes are not hybridized to their complementary sequence on the amplified target nucleotide sequence, then the distance between the donor fluorophore and the acceptor fluorophore is too great for resonance energy transfer to occur. Thus, the acceptor fluorophore and the donor fluorophore are not in resonance energy transfer relationship and excitation of the donor fluorophore will not produce a detectable fluorescent emission by the acceptor fluorophore.

Preferred nucleotide sequences that can be used for the two fluorescently labeled nucleotide probes include respectively SEQ ID NOs: 5 and 6 and SEQ ID NOs: 7 and 8. Pairs of nucleotide sequences that are complementary and/or substantially similar to SEQ ID NOs: 5 and 6 and SEQ ID NOs: 7 and 8, are also preferred.

The nucleic acids of the inventive oligonucleotide primers and hybridization probes may be made by methods well known in the art, such as chemical synthesis. The inventive oligonucleotide primers and hybridization probes may be synthesized manually or by machine. They may also be synthesized by recombinant methods using products incorporating viral and bacterial promoters.

In accordance with a method of the present invention, the oligonucleotide primers and hybridization probes can be used in a polymerase chain reaction (PCR) assay to detect and identify H. capsulatum. In the method, a sample suspected of harboring H. capsulatum is obtained. The sample may be concentrated and subjected to a procedure that partially purifies the nucleic acids in the sample.

The sample suspected of harboring H. capsulatum is then subjected to polymerase chain reaction (PCR) amplification. In PCR amplification, at least a portion of the sample is contacted with oligonucleotide primers. The oligonucleotide primers include nucleic acid sequences that are specific to H. capsulatum and that can be used to amplify a target nucleotide sequence. The target nucleotide sequence is defined by contiguous nucleotides from the ITS-1 region and 5.8s region of H. capsulatum. Preferably, the oligonucleotide primers include a pair of nucleic acid sequences that flank the target nucleotide sequence of H. capsulatum which is to be amplified. One preferred pair of nucleic acid sequences includes SEQ ID NOs: 1 and 2. Another preferred pair of nucleic acid sequences includes SEQ ID NOs: 3 and 4. Pairs of nucleic acid sequences that are complementary and/or substantially similar to SEQ ID NOs: 1 and 2 and SEQ ID NOs: 3 and 4, are also preferred.

PCR amplification is then conducted on the resulting mixture using a temperature program and for a number of thermal cycles sufficient to amplify the target nucleotide sequence of H. capsulatum, if present. The PCR amplification can be carried out in any commercially available PCR thermal cycling apparatus. Preferably, the PCR amplification is performed using rapid temperature cycling techniques. Rapid temperature cycling techniques use a high surface area-to-volume sample container, such as a capillary tube, to contain the reaction amplification sample. The use of a high surface-area-to-volume sample container allows for rapid temperature response and temperature homogeneity throughout the sample. Rapid temperature cycling is contrasted to conventional temperature cycling in that 30 cycles of amplification can be completed in 15 minutes and the resulting PCR amplification products contain fewer side products. Thus, with rapid temperature cycling techniques the required times for amplification are reduced approximately ten-fold, and specificity is improved.

The amplified target nucleotide sequence, if present, is then detected using an oligonucleotide hybridization probe in accordance. The oligonucleotide hybridization probe includes a nucleic acid sequence that is capable of hybridizing to the amplified DNA of H. capsulatum. Preferably, the oligonucleotide hybridization probe includes a pair of nucleic acid sequences that are labeled with a fluorescence resonance energy transfer (FRET) pair. Preferred, pairs of nucleic acid sequences that can be fluorescently labeled include respectively SEQ ID NOs: 5 and 6 and SEQ ID NOs: 7 and 8. Pairs of nucleic acid sequences that are complementary and/or substantially similar to SEQ ID NOs: 5 and 6 and SEQ ID NOs: 7 and 8, are also preferred.

When the detection method produces a result indicating that target nucleotide sequence amplified by the oligonucleotide primers is present, it is concluded that the original sample contains H. capsulatum. Conversely, if no evidence of the target nucleotide sequence is detected, it is concluded that sample is free of H. capsulatum.

In preferred embodiment of the present invention, the polymerase chain reaction (PCR) amplification step and the detection step of the method are performed essentially simultaneously. Preferably, the essentially simultaneous PCR amplification step and the detection step are performed in an apparatus that includes a rapid temperature cycler component and a fluorescent detection component. An example of such a device is described in U.S. Pat. No. 6,140,540, the disclosure of which is incorporated herein by reference. The device comprises a chamber, a heater, a fan, and a carousel. The fan is mounted in the device and in air flow communication with the chamber. The carousel is rotatably mounted in the chamber and holds a plurality of sample vessels. The sample vessels used in conjunction with the device comprise an optically transparent material. The device further comprises a light emitting source and a light detector. The light emitting source is mounted in the chamber and positioned to illuminate at least one of the sample vessels. The light detector is mounted in the chamber and positioned to measure fluorescence from at least one of the sample vessels. A preferred device that includes a rapid cycler component and fluorescent detection component is commercially available from Roche Molecular Biochemicals, of Indianapolis, Ind. under the trade name LIGHTCYCLER.

The present invention is further directed to a kit for identifying and detecting H. capsulatum in a biological sample by means of a polymerase chain reaction (PCR) assay. The kit includes at least one pair of oligonucleotide primers and at least one pair of oligonucleotide hybridization probes. The pair of oligonucleotide primers includes nucleic acid sequences that are specific to H. capsulatum and that can be used to amplify a target nucleotide sequence, which is defined by contiguous nucleotides from the ITS-1 region and 5.8s region of the DNA of H. capsulatum.

In one embodiment of the present invention, the kit comprises a pair of oligonucleotide primers that include nucleic acid sequences having SEQ ID NOs: 1 and 2 and a pair of oligonucleotide hybridization probe that includes nucleic acid sequences having SEQ ID NOs: 5 and 6. The oligonucleotide hybridization probes are preferably labeled respectively with a donor fluorophore and an acceptor fluorophore. More preferably, the oligonucleotide hybridization probe that includes SEQ ID No. 5 is labeled at the 3′-end of the probe with Fluorescein (5-FITC), and the oligonucleotide hybridization probe that includes SEQ ID NO. 6 is labeled at the 3′-end of the probe with LightCycler Red 640.

In another preferred embodiment of the present invention, the kit comprises a pair of oligonucleotide primers that include nucleic acid sequences having SEQ ID NOs: 3 and 4, and a pair of oligonucleotide hybridization probe that includes nucleic acid sequences having SEQ ID NOs: 7 and 8. The oligonucleotide hybridization probes are preferably labeled respectively with a donor fluorophore and an acceptor fluorophore. More preferably, the oligonucleotide hybridization probe that includes SEQ ID No. 7 is labeled at the 3′-end of the probe with Fluorescein (5-FITC), and the oligonucleotide hybridization probe that includes SEQ ID NO. 8 is labeled at the 3′-end of the probe with LightCycler Red 640.

Alternatively, the kit may also contain one or all of the reagents necessary to begin the PCR amplification reaction and fluorescent detection of the oligonucleotide probes.

### EXAMPLE

The following example illustrates use of oligonucleotide primers and oligonucleotide hybridization probes in accordance with the present invention for the amplification and detection of H. capsulatum. The example used a LIGHTCYCLER polymerase chain reaction device, which was commercially available from Roche Molecular Biochemicals of Indianapolis, Ind., for hybridization and probe melting studies. The oligonucleotide primers and hybridization probes were tested against a battery of 86 clinical fungal isolates, 20 of which were H. capsulatum. All H. capsulatum isolates had been confirmed with the H. capsulatum ACCUPROBE (Gen-Probe, Inc. San Diego, Calif.). The remaining fungi tested include B. dermatitidis, to test a genetically related fungus, and a variety of other fungi routinely isolated in a mycology laboratory. The other fungi tested included Coccidioides immitis, Aspergillus species, Candida species, as well as a variety of yeast and hyaline and dematiacecous molds.

Materials and Methods

All of the archived isolates of H. capsulatum and B. dermatitidis were studied. In addition, other routine fungal isolates were also studied. A complete list of the isolates that were tested is included in Table 1. The molds included in this study were identified by traditional methods, predominantly microscopic and colonial morphology. In addition to this, the identification of H. capsulatum, B. dermatitidis, and C. immitiswas confirmed by the respective H. capsulatum, B. dermatitidis, and C. immitisAccuProbes® (Gen-Probe, Inc., San Diego, Calif.). The yeast isolates studied were also identified by traditional methods, using the Vitek yeast identification card (biomerieux, St Louis, Mo.), supplemental biochemical testing when necessary, and colony morphology.

All isolates of H. capsulatum and B. dermatitidis were retrieved from frozen storage at 4° C. and cultivated on Potato Dextrose agar. The other fungi tested were obtained after they were routinely identified from clinical specimens in the mycology laboratory. The nucleic acid was extracted from these organism and PCR with specific hybridization detection probes was carried out in the LightCycler instrument (Roche Molecular Biochemicals, Indianapolis, Ind.) as described below.

Fungal DNA Extraction from Culture Isolates

A 5 mm squared portion of fungus was removed from each culture plate. If the culture was a mold, the adherent agar was removed. If the isolate was a yeast, adherent agar was not a problem. The fungi were extracted in 500 μL of lysis buffer [3] containing 1% Triton X-100, 0.5% Tween 20, 10 mM Tris-HCl (pH 8.0) and 1 mM EDTA and incubated in a heating block at 100° C. for 30 minutes. The fluid was stored at −20° C. for future testing.

PCR and Hybridization Probe Detection

The oligonucleotide primers and hybridization probes used had the following sequences:

Forward Primer:
5′-TTGTCTACCGGACCTG-3′
Reverse Primer:
5′-TTCTTCATCGATGTCGGAAC-3′
Hybridization Probe 1:
5′-ACGATTGGCCTCTGAGC-FITC-3′
Hybridization Probe 2:
5′-LCRd640-GAGAGCGATAATAATCCAGTCAAAAC-phosphate-3′

The target of amplification and detection was a portion of the internal transcribed spacer region (ITS-1) of H. capsulatum. The concentrations of primers and probes are given in Table 2. The reaction volume of 20 μL was a mixture 5 μL extracted target DNA and 15 μL of Hybridization Probe master mix (Roche). These were placed together in a LightCycler capillary tube. The LightCycler PCR parameters were used. A suspension of TE buffer was used as the negative control.

The presence of amplified DNA was measured by detection of energy emitted at 640 nm. The temperature at which the hybridization probes disassociated from the target DNA probe hybridization sites was determined by melting curve analysis, as provided for by the LightCycler software. This served as an independent indicator of the specificity of hybridization.

TABLE 1

| | Test Results |
|---|---|
| Organisms Tested by the | (negative (−) |
| Histoplasma capsulatum | or positive |
| LightCycler Assay | (+)) |
| Histoplasma capsulatum (20 | + |
| isolates) | |
| Blastomyces dermatitidis (6 | − |
| isolates) | |
| At least one isolate of: | |
| Candida albicans | − |
| Candida rugosa | − |
| Candida lusitaniae | − |
| Candida parapsilosis | − |
| Candida glabrata | − |
| Candida tropicalis | − |
| Candida rugosa | − |
| Candida guilliermondii | − |
| Cryptococcus neoformans | − |
| Cryptococcus terreus | − |
| Cryptococcus laurentii | − |
| Cryptococcus albidus | − |
| Rhizopus species | − |
| Mucor species | − |
| Microsporium species | − |
| Trichophyton verricosum | − |
| Trichophyton species | − |
| Aspergillus versicolor | − |
| Aspergillus fumigatus | − |
| Aspergillus niger | − |
| Aspergillus flavus | − |
| Aspergillus species | − |
| Chrysosporium species | − |
| Scedosporium apiospermum | − |
| Scytalidium species | − |
| Scopulariopsis species | − |
| Fusarium species | − |
| Coccidioides immitis | − |
| Cladosporium species | − |
| Sporothrix schenckii | − |
| Arthrographis species | − |
| Paecilomyces species | − |
| Phialamonium species | − |
| Pithomyces species | − |
| Ochroconis species | − |
| Epicoccum species | − |
| Trichothecium species | − |
| Penicillium species | − |
| Exophiala jenselmei | − |
| Aureobacterium species | − |
| Chrysonilia | − |
| Hormonema species | − |
| Trichosporon beigelli | − |
| Rhodotorula species | − |
| Phaeoannellomyces wernekii | − |
| Blastoschizomyces species | − |

TABLE 2
Composition of the amplification reaction mixtures
Master mix for the Histoplasma capsulatum detection

| Reagent | Final concentration |
|---|---|
| H. cap Forward primer | 0.5 μM |
| H. cap Reverse primer | 0.5 μM |
| H. cap Probe 1 | 0.2 μM |
| H. cap Probe 2 | 0.4 μM |
| MgCl₂ | 4.0 μM |

Results

The described H. capsulatum PCR/hybridization probes detected only H. capsulatum, giving 100% sensitivity and 100% specificity. No hybridization melt curves were detected for any other fungi tested. Eighteen of Twenty isolates of H. capsulatum tested had an average hybridization melting temperature of 51.35° C. (range 50.94-51.90° C.). Two isolates, however, had an average hybridization melting temperature of 42.93° C. (range 42.78-43.08° C.). This lower melting temperature is believed to be caused by a mutation at the probe hybridization site.

From the above description of the invention, those skilled in the art will perceive improvements, changes and modifications. Such improvements, changes and modifications within the skill of the art are intended to be covered by the appended claims.

1. A nucleic acid specific for use in identifying Histoplasma capsulatum, said nucleic acid comprising a sequence being selected from the group consisting of SEQ ID NOs: 1-8, a sequence complementary to SEQ ID NOs: 1-8, a sequence substantially similar to SEQ ID NOs: 1-8, a sequence substantially similar to a sequence complementary to SEQ ID NOs: 1-8, and a fragment of SEQ ID NOs: 1-8, a sequence complementary to SEQ ID NOs: 1-8, or a sequence substantially similar to SEQ ID NOs: 1-8, a sequence substantially similar to a sequence complementary to SEQ ID NO: 1-8 that specifically hybridizes to Histoplasma capsulatum.

2. An oligonucleotide primer specific for use in identifying Histoplasma capsulatum, said oligonucleotide primer comprising a nucleic acid sequence selected from the group consisting of SEQ ID NOs: 1-4, a sequence complementary to SEQ ID NOs: 1-4, a sequence substantially similar to SEQ ID NOs: 1-4, a sequence substantially similar to a sequence complementary to SEQ ID NOs: 1-4, and a fragment of SEQ ID NOs: 1-4, a sequence complementary to SEQ ID NOs: 1-4, or a sequence substantially similar to SEQ ID NOs: 1-4, a sequence substantially similar to a sequence complementary to SEQ ID NO: 1-4 that specifically hybridizes to Histoplasma capsulatum.

3. The oligonucleotide primer of claim 2, wherein the oligonucleotide primer comprises a pair of nucleic acid sequences which flank a target nucleotide sequence of H. capsulatum.

4. The oligonucleotide primer of claim 3, wherein the pair of nucleic acid sequences includes SEQ ID NO: 1 and SEQ ID NO: 2.

5. The oligonucleotide primer of claim 3, wherein the pair of nucleic acid sequences includes SEQ ID NO: 3 and SEQ ID NO: 4.

6. An oligonucleotide hybridization probe for identifying Histoplasma capsulatum, said probe comprising a nucleic acid sequence selected from the group consisting of SEQ ID NOs: 5-8, a sequence complementary to SEQ ID NOs: 5-8, a sequence substantially similar to SEQ ID NOs: 5-8, a sequence substantially similar to a sequence complementary to SEQ ID NOs: 5-8, and a fragment of SEQ ID NOs: 5-8, a sequence complementary to SEQ ID NOs: 5-8, or a sequence substantially similar to SEQ ID NOs: 5-8, a sequence substantially similar to a sequence complementary to SEQ ID NOs: 5-8 that specifically hybridizes to Histoplasma capsulatum.

7. The oligonucleotide hybridization probe of claim 6, wherein said nucleic acid sequence is labeled with a detectable moiety.

8. The oligonucleotide hybridization probe of claim 7 wherein the detectable moiety is a fluorescent label.

9. The oligonucleotide hybridization probe of claim 6 wherein the oligonucleotide hybridization probe is detectable by fluorescence resonance energy transfer.

10. A method of detecting the presence of Histoplasma capsulatum in a sample, said method comprising the steps of:
providing a sample suspected of including Histoplasma capsulatum;
amplifying a Histoplasma capsulatum target nucleotide sequence using an oligonucleotide primer comprising a nucleic acid sequence selected from the group consisting of SEQ ID NOs: 1-4, a sequence complementary to SEQ ID NOs: 1-4, a sequence substantially similar to SEQ ID NOs: 1-4, a sequence substantially similar to a sequence complementary to SEQ ID NOs: 1-4, and a fragment of SEQ ID NOs: 1-4, a sequence complementary to SEQ ID NOs: 1-4, or a sequence substantially similar to SEQ ID NOs: 1-4, a sequence substantially similar to a sequence complementary to SEQ ID NO: 1-4 that specifically hybridizes to Histoplasma capsulatum; and
contacting the amplified target nucleic acid with an oligonucleotide hybridization probe which is capable of hybridizing to the amplified target nucleotide sequence.

11. The method of claim 10 wherein said amplifying step is performed using polymerase chain reaction in a rapid temperature cycler.

12. The method of claim 11 wherein the amplified target nucleotide sequence is detected by fluorescence.

13. The method of claim 12 wherein the amplified target nucleotide sequence is detected by at least on fluorescently labeled oligonucleotide hybridization probe.

14. The method of claim 10 wherein the amplified target nucleotide sequence is detected by two oligonucleotide hybridization probes, each labeled with a fluorescent moiety, such that when both probes are hybridized to the target nucleotide sequence, fluorescence resonance energy transfer occurs between the fluorescent moieties.

15. The method of claim 13 wherein the oligonucleotide hybridization probe comprises a nucleic acid sequence selected from the group consisting of SEQ ID NOs: 5-8, a sequence complementary to SEQ ID NOs: 5-8, a sequence substantially similar to SEQ ID NOs: 5-8, a sequence substantially similar to a sequence complementary to SEQ ID NOs: 5-8, and a fragment of SEQ ID NOs: 5-8, or a sequence complementary to SEQ ID NOs: 5-8, a sequence substantially similar to SEQ ID NOs: 5-8, a sequence substantially similar to a sequence complementary to SEQ ID NOs: 5-8 that specifically hybridizes to Histoplasma capsulatum.

16. The method of claim 14 wherein the pair of oligonucleotide hybridization probes comprises, respectively, SEQ ID NO: 5 and SEQ ID NO: 6.

17. The method of claim 14 wherein the pair of oligonucleotide hybridization probes comprises, respectively, SEQ ID NO: 7 and SEQ ID NO: 8.

18. A method of detecting the presence of Histoplasma capsulatum in a sample, said method comprising the steps of:
providing a sample suspected of including Histoplasma capsulatum;
amplifying a target nucleotide sequence that comprises contiguous nucleotides from the ITS-1 and 5.8s gene region of Histoplasma capsulatum; and
contacting the amplified target nucleotide sequence with an oligonucleotide hybridization probe comprising a nucleic acid sequence selected from the group consisting of SEQ ID NOs: 5-8, a sequence complementary to SEQ ID NOs: 5-8, a sequence substantially similar to SEQ ID NOs: 5-8, a sequence substantially similar to a sequence complementary to SEQ ID NOs: 5-8, and a fragment of SEQ ID NOs: 5-8, a sequence complementary to SEQ ID NOs: 5-8, a sequence substantially similar to SEQ ID NOs: 5-8, or a sequence substantially similar to a sequence complementary to SEQ ID NOs: 5-8 that specifically hybridizes to Histoplasma capsulatum.

19. The method of claim 18 wherein said amplifying step is performed using polymerase chain reaction in a rapid temperature cycler.

20. The method of claim 18 wherein the amplified target nucleotide sequence is detected by fluorescence.

21. The method of claim 18 wherein the amplified target nucleotide sequence is detected by at least on fluorescently labeled oligonucleotide hybridization probe.

22. The method of claim 21 wherein the amplified target nucleotide sequence is detected by two oligonucleotide hybridization probes, each labeled with a fluorescent moiety, such that when both probes are hybridized to the target nucleotide sequence, fluorescence resonance energy transfer occurs between the fluorescent moieties.

23. The method of claim 18, wherein the target nucleotide sequence is amplified using an oligonucleotide primer comprising a nucleic acid sequence selected from the group consisting of SEQ ID NOs: 1-4, a sequence complementary to SEQ ID NOs: 1-4, a sequence substantially similar to SEQ ID NOs: 1-4, a sequence substantially similar to a sequence complementary to SEQ ID NOs: 1-4, and a fragment of SEQ ID NOs: 1-4, a sequence complementary to SEQ ID NOs: 1-4, a sequence substantially similar to SEQ ID NOs: 1-4, or a sequence substantially similar to a sequence complementary to SEQ ID NO: 1-4 that specifically hybridizes to Histoplasma capsulatum.

24. A kit for use in detecting Histoplasma capsulatum, said kit comprising:
a primer that includes a nucleic acid sequence selected from the group consisting of SEQ ID NOs: 1-4, a sequence complementary to SEQ ID NOs: 1-4, a sequence substantially similar to SEQ ID NOs: 1-4, a sequence substantially similar to a sequence complementary to SEQ ID NOs: 1-4, and a fragment of SEQ ID NOs: 1-4, a sequence complementary to SEQ ID NOs: 1-4, a sequence substantially similar to SEQ ID NOs: 1-4, or a sequence substantially similar to a sequence complementary to SEQ ID NO: 1-4 that specifically hybridizes to Histoplasma capsulatum; and
a nucleic acid hybridization probe that includes a nucleic acid sequence selected from the group consisting of SEQ ID NOs: 5-8, a sequence complementary to SEQ ID NOs: 5-8, a sequence substantially similar to SEQ ID NOs: 5-8, a sequence substantially similar to a sequence complementary to SEQ ID NOs: 5-8, and a fragment of SEQ ID NOs: 5-8, a sequence complementary to SEQ ID NOs: 5-8, or a sequence substantially similar to SEQ ID NOs: 5-8, a sequence substantially similar to a sequence complementary to SEQ ID NOs: 5-8 that specifically hybridizes to Histoplasma capsulatum.
